(19) United States
(12) Patent Application Publication
(10) Pub. No.: US 2007/0260151 A1
(43) Pub. Date: Nov. 8, 2007

(54) METHOD AND DEVICE FOR FILTERING, SEGMENTING, COMPRESSING AND CLASSIFYING OSCILLATORY SIGNALS

(76) Inventor: Gari D. Clifford, Boston, MA (US)

Correspondence Address: MCGUIREWOODS, LLP, 1750 TYSONS BLVD, SUITE 1800, MCLEAN, VA, 22102, US

(21) Appl. No.: 11/470,506

(22) Filed: Sep. 6, 2006

Related U.S. Application Data
(60) Provisional application No. 60/746,315, filed on May 3, 2006.
(60) Provisional application No. 60/799,327, filed on May 11, 2006.

(51) Int. Cl. A61B 5/04

(52) U.S. Cl. 600/509

(57) ABSTRACT

A method, system, and computer readable medium executable on a computer for at least one of filtering, segmenting, compressing and classifying an ECG or similar signal includes the steps of fitting a nonlinear signal model to the signal using an optimization algorithm, such as nonlinear least squares, and determining features in the nonlinear signal model.

### CROSS-REFERENCE TO RELATED APPLICATIONS

This application claims priority under 35 U.S.C. §119(e) to provisional U.S. Patent Application No. 60/746,315, filed on May 3, 2006; and provisional U.S. Patent Application No. 60/799,327, filed on May 11, 2006 the disclosures of which are expressly incorporated by reference herein in their entireties.

### BACKGROUND OF THE INVENTION

1. Field of the Invention

The invention is directed to a method for filtering, segmenting, compressing and/or classifying oscillatory signals in a morphology-specific manner and a device for its practice and, in particular, for filtering, segmenting, compressing and/or classifying physiological signals, such as ECG signals, in a subject-specific manner.

2. Related Art

Currently, no method exists for accurately analyzing individually specific oscillatory signals, such as physiological signals. For example, ECG analysis algorithm development has reached a plateau in the last 10 years, despite significant advances in computational power.

Conventional physiological signal filtering, such as ECG filters have been limited by their generic applicability in that they use only a vague knowledge of the expected frequency band of interest and use almost no information concerning either the general morphology of an ECG, or a patient specific template.

This is in part due to the inability of conventional filters to remove in-band

Hz) noise (the frequency range can be as high as 500 Hz and as low as 0.02 Hz).

Adaptive filters have been proposed which require another reference signal, or some ad-hoc generic model of the signal as an input. Adaptive filters have been used to remove in-band noise, but the nonlinear and unpredictable characteristics of these filters can lead to significant and unreliable distortions of the clinical parameters (QT interval, ST level, QRS axis etc.) in the filtered ECG. Part of the problem is due to the fact that the adaptive filter qualities change based on the signal, and because the model for the ECG is poor. Recent techniques that can remove in-band noise in certain circumstances (such as Independent Component Analysis—ICA), have been unreliable since they too change based upon the varying qualities of the signal. Furthermore, they are not constrained by any model of the ECG.

Also, a number of algorithms for automated QT analysis are based on threshold methods that attempt to predict the end of the T wave as the point where the T wave crosses a predetermined threshold. However, the known methods have resulted in less than satisfactory results because the QT interval cannot be accurately determined.

Accordingly, there is a need for a method of constraining analysis techniques for physiological signals by a realistic model in order to overcome these problems.

One example of a specific application in which such a model is desirable is in the monitoring of patients for use in the studies related to the development of new drugs. More specifically, the development of new drugs by the pharmaceutical industry is a costly and lengthy process, with the time from concept to final product typically lasting ten years. Perhaps the most critical stage of this process is the phase one study, where the drug is administered to humans for the first time. During this stage, each subject is carefully monitored for any unexpected adverse effects that may be brought about by the drug. Of particular interest is the electrocardiogram (ECG) of the patient, which provides detailed information about the state of the patient's heart. By examining the ECG signal in detail, it is possible to derive a number of informative measurements from the characteristic ECG waveform. These characteristics can then be used to assess the medical well-being of the patient, and more importantly, detect any potential side effects of the drug on the cardiac rhythm. The most important of these measurements is the QT interval. In particular, drug-induced prolongation of the QT interval can result in a very fast, abnormal heart rhythm known as torsade de pointes, which is often followed by sudden cardiac death.

In practice, QT interval measurements are carried out manually by trained ECG analysts. This is an expensive and time-consuming process, which is susceptible to mistakes by the analysts and provides no associated degree of confidence (or accuracy) in the measurements. This problem was highlighted in the case of the antihistamine terfenadine, which has the side-effect of significantly prolonging the QT interval in a number of patients. Unfortunately, this side-effect was not detected in the clinical trials and only came to light after a number of people had unexpectedly died while taking the drug.

The timing between the onset and offset of particular features of the ECG (referred to as an interval) is of great importance since it provides a measure of the state of the heart and can indicate the presence of certain cardiac conditions. The three most important intervals in the ECG waveform are the PR interval, QRS width and the QT interval (see FIG. 5). The QT interval is defined as the time from the start of the QRS complex to the end of the T wave, and corresponds to the total duration of electrical activity (both depolarization and repolarization) in the ventricles. Similarly, the PR interval is defined as the time from the start of the P wave to the start of the QRS complex, and corresponds to the time from the onset of atrial depolarization to the onset of ventricular depolarization. Changes in the QT interval are currently the gold standard for evaluating the effects of drugs on ventricular repolarization. In addition, changes in the PR interval can indicate the presence of special cardiac conditions such as atrioventricular block. Thus, the accurate measurement and assessment of the QT and PR intervals is of paramount importance, particularly for the assessment and validation of new drugs in clinical trials. The measurement of the QT interval is complicated by the fact that a precise mathematical definition of the end of the T wave does not exist. Thus, T wave end measurements are inherently subjective and the resulting QT interval measurements often suffer from a high degree of inter- and intra-analyst variability.

The Expert Working Group (Efficacy) of the International Conference on Harmonization of Technical Requirements for Registration of Pharmaceuticals for Human Use (ICH) developed and gave its final (“step 4”) endorsement to a set of guidelines (ICH E14) for clinical evaluation of QT/QTc interval prolongation and proarrhythmic potential for non-antiarrhythmic drugs. It is clear from the text of the ICH E14 guidelines that regulatory agencies are currently unconvinced of the reliability of automatic QT interval measurements.

Hence, there also is a need for an automated ECG interval analysis system and method, which provides robust and consistent measurements (together with an associated degree of confidence in each measurement) for validation of new drugs in clinical trials.

### SUMMARY OF THE INVENTION

The invention meets the foregoing needs and provides a method and system to accurately and quickly analyze oscillatory signals such as ECG or other physiological signals, which results in superior data research, such as for new drug development, and other advantages apparent from the discussion herein.

Accordingly, an alternative filtering paradigm that uses a patient specific model of an ECG signal, yet requires no prior knowledge of the morphology and only one channel of the ECG.

The invention may use a realistic model of the ECG to aid filtering and signal representation. Moreover, the method may be tuned to an individual's morphology, rather than using a standard global set of basis functions trained on a population set. Each beat is analyzed in isolation, on a beat-by-beat basis, allowing the segmentation of every part of the signal. No heuristics are required and the functions that model the signal are completely interchangeable—any functions may be used. The choice of Gaussians allows for a statistically accurate determination of the end and start of each wave in the ECG. The Fourier Transform of a Gaussian is another Gaussian, so the frequency content of the ECG can be calculated with greater accuracy (and analytically).

With the invention, the ECG may be compressed into only 18 parameters per beat (width, height, and location of each of the 6 Gaussians). There is no noise in the ECG model, so fitting the parameters to the ECG gives a very smooth representation. Classification may be performed on a stable and minimal number of functions that are sensitive to morphology changes. The parameters of the model are all implicitly related to each other, and therefore, classification of the signal from the fitted parameters allows one to detect a subtle change in the signal that manifests as small changes across each wave in the P-QRS-T morphology. The error in the model-fit provides a confidence in classification of measurement and may include an ability to reject noisy segments from confidence indices.

The invention may be used to model any physiological signal—and is not confined solely to an ECG signal—but may be blood pressure, central venous pressure, pulmonary arterial pressure, pulse oximetry (SAO2), cardiac sounds, or even non-cardiovascular signals such as EEG K-complexes, muscular activity, neural activity, acoustic waveforms, and speech waveforms. The invention may also be applied to other fields where such modeling is desirable, such as stress vibrations, radio transmissions, sensor signals, or other signals characterized by oscillations at specific frequencies, and/or contaminated by in-band noise.

Accordingly, the invention may be implemented in a number of ways. In one aspect of the invention, a computer-implemented method for at least one of filtering, segmenting, compressing, and classifying an ECG signal includes the steps of: obtaining an ECG signal, storing the ECG signal, generating a nonlinear signal model based on the ECG signal, fitting the nonlinear signal model to the ECG signal based on an optimization algorithm, determining at least one feature of the ECG with the nonlinear signal model, and outputting the at least one feature of the ECG based on the nonlinear signal model.

An adaptive filter may use the above-noted method. The filter may operate on a beat-by-beat basis. The step of generating a nonlinear signal model may correspond to modeling at least one segment of interest of the ECG signal selected from the group consisting of a QT interval, a Q-wave onset (PQ-junction), a T-wave offset, a T-wave height, a U-wave detection and characterization, and a T-wave asymmetry of the ECG signal. The generating step may include the use of Gaussian descriptors and/or the optimization algorithm in the fitting step may include least squares optimization. The step of determining at least one feature may include determining at least one of or each one of a QT interval, a Q-wave onset (PQ-junction), a T-wave offset, a T-wave height, a U-wave detection and characterization, and a T-wave asymmetry of the ECG signal. The step of determining at least one feature may include determining the locations of the P, Q, R, S and T features of each beat of the ECG signal. The generating a non-linear signal model step further may include the steps of: locating at least one fiducial point in the ECG signal, performing a temporal average of time series segments around the at least one fiducial point, accepting features inside a threshold, and determining the symmetry of the features that are accepted. The generating step further may include the steps of: fitting the model to the features, and rejecting model fit when the model exceeds a threshold.

According to another aspect of the invention, a computer readable medium executable on a computer for at least one of filtering, segmenting, compressing, and classifying an oscillatory physiological signal, may execute some or all of the method steps described above.

According to yet another aspect of the invention, a computer system for at least one of filtering, segmenting, compressing and classifying an oscillatory physiological signal includes: an input to receive an ECG signal, a storage device responsive to the input to store the ECG signal, a processor to generate a nonlinear signal model based on the ECG signal, fit the nonlinear signal model to the ECG signal based on an optimization algorithm, and determine at least one feature of the ECG with the nonlinear signal model, and an output device to output the at least one feature of the ECG based on the nonlinear signal model.

The nonlinear signal model may correspond to at least one segment of interest of the ECG signal. The nonlinear signal model may include Gaussian descriptors and/or the optimization algorithm may include least squares optimization. The at least one feature may include at least one of a QT interval, a Q-wave onset (PQ-junction), a T-wave offset, a T-wave height, a U-wave detection and characterization, and a T-wave asymmetry of the ECG signal. The at least one feature may include at least one of the locations of the P, Q, R, S and T features of each beat of the ECG signal.

In yet another aspect of the invention, a computer system for at least one of filtering, segmenting, compressing and classifying an ECG signal, includes: means for receiving an ECG signal, means for storing the ECG signal, means for generating a nonlinear signal model based on the ECG signal, fitting the nonlinear signal model to the ECG signal based on an optimization algorithm, and determining at least one feature of the ECG with the nonlinear signal model, and means for outputting the at least one feature of the ECG based on the nonlinear signal model.

The nonlinear signal model may correspond to at least one segment of interest of the ECG signal. Again, the nonlinear signal model may include Gaussian descriptors and/or the optimization algorithm may include least squares optimization. The at least one feature may include at least one of a QT interval, a Q-wave onset (PQ-junction), a T-wave offset, a T-wave height, a U-wave detection and characterization, and a T-wave asymmetry of the ECG signal. The at least one feature may include at least one of the locations of the P, Q, R, S and T features of each beat of the ECG signal.

In yet a further aspect of the invention, a method for correlated source separation of biomedical signals includes the steps of: obtaining a biomedical signal, storing the biomedical signal, generating a nonlinear signal model based on the biomedical signal, fitting the nonlinear signal model to the biomedical signal based on an optimization algorithm, determining at least one feature of the biomedical with the nonlinear signal model, and outputting the at least one feature of the biomedical based on the nonlinear signal model.

Again, an adaptive filter, computer readable medium, or a computer system may use the above-noted method for correlated source separation of biomedical signals, and may operate on a beat-by-beat basis. The generating step may include the use of Gaussian descriptors and/or the optimization algorithm in the fitting step may include least squares optimization. The generating step further may include the steps of: locating at least one fiducial point in the biomedical signal, performing a temporal average of time series segments around the at least one fiducial point, accepting features inside a threshold, and determining the symmetry of the features that are accepted. The generating step further may include the steps of: fitting the model to the features, and rejecting model fit when the model exceeds a threshold. The biomedical signal may be a physiological signal such as an ECG signal.

The benefits of the invention may include: increased accuracy of clinical parameter derivation (such as QT interval, ST level, QRS axis); more sensitive diagnostics; automated analysis (saving costs on human oversight); increased sensitivity to abnormal beats and rhythms; ability to reject noisy segments and produce confidence indices; and a high compression rate—that allows for rapid and cheap transmission of data, and lower storage requirements.

Additional features, advantages, and embodiments of the invention may be set forth or apparent from consideration of the following detailed description, drawings, and claims. Moreover, it is to be understood that both the foregoing summary of the invention and the following detailed description are exemplary and intended to provide further explanation without limiting the scope of the invention as claimed.

### BRIEF DESCRIPTION OF THE DRAWINGS

The accompanying drawings, which are included to provide a further understanding of the invention, are incorporated in and constitute a part of this specification, illustrate embodiments of the invention and together with the detailed description serve to explain the principles of the invention. No attempt is made to show structural details of the invention in more detail than may be necessary for a fundamental understanding of the invention and the various ways in which it may be practiced. In the drawings:

FIG. 1 is a flowchart schematically illustrating a generalized method for constructing a model fit signal according to the principles of the invention;

FIG. 2 shows an original (clean) graphed ECG signal, a model fit signal constructed according to the principles of the invention and the residual error between the two signals;

FIG. 3 shows a model fit to an ECG signal of the invention under high noise conditions. The underlying signal before noise was added and is shown. Note that the model fit preserves the overall morphology and placement of the onset and offset of the main features;

FIG. 4 shows a ST-elevated waveform and model fit constructed according to the principles of the invention;

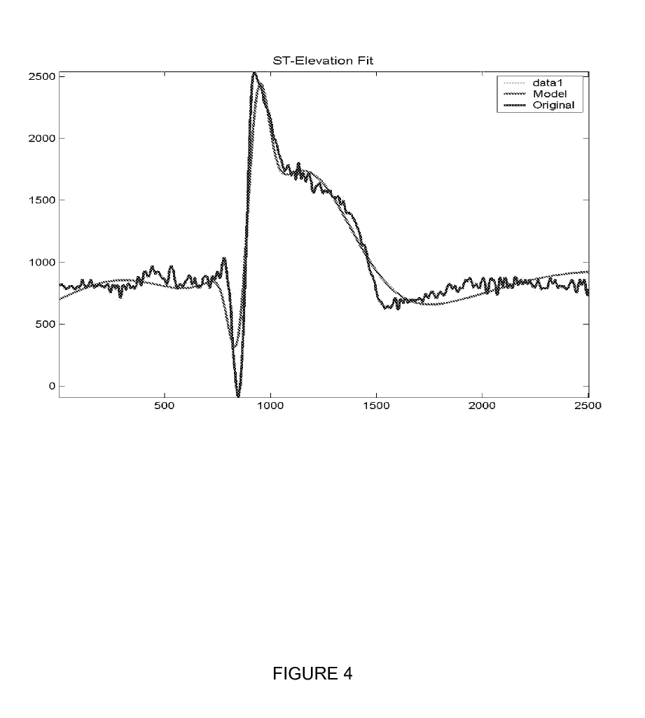

FIG. 5 shows a typical ECG with labels relevant to QT analysis; and

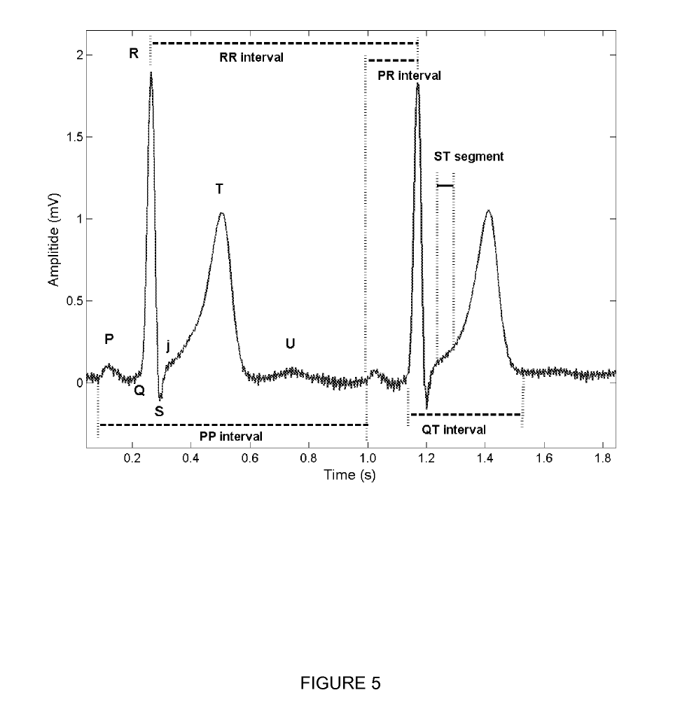

FIG. 6 shows a schematic of a computer system constructed according to the principles of the invention for use with the method of FIG. 1.

### DETAILED DESCRIPTION OF THE INVENTION

The embodiments of the invention and the various features and advantageous details thereof are explained more fully with reference to the non-limiting embodiments and examples that are described and/or illustrated in the accompanying drawings and detailed in the following description. It should be noted that the features illustrated in the drawings are not necessarily drawn to scale, and features of one embodiment may be employed with other embodiments as the skilled artisan would recognize, even if not explicitly stated herein. Descriptions of well-known components and processing techniques may be omitted so as to not unnecessarily obscure the embodiments of the invention. The examples used herein are intended merely to facilitate an understanding of ways in which the invention may be practiced and to further enable those of skill in the art to practice the embodiments of the invention. Accordingly, the examples and embodiments herein should not be construed as limiting the scope of the invention, which is defined solely by the appended claims and applicable law. Moreover, it is noted that like reference numerals represent similar parts throughout the several views of the drawings.

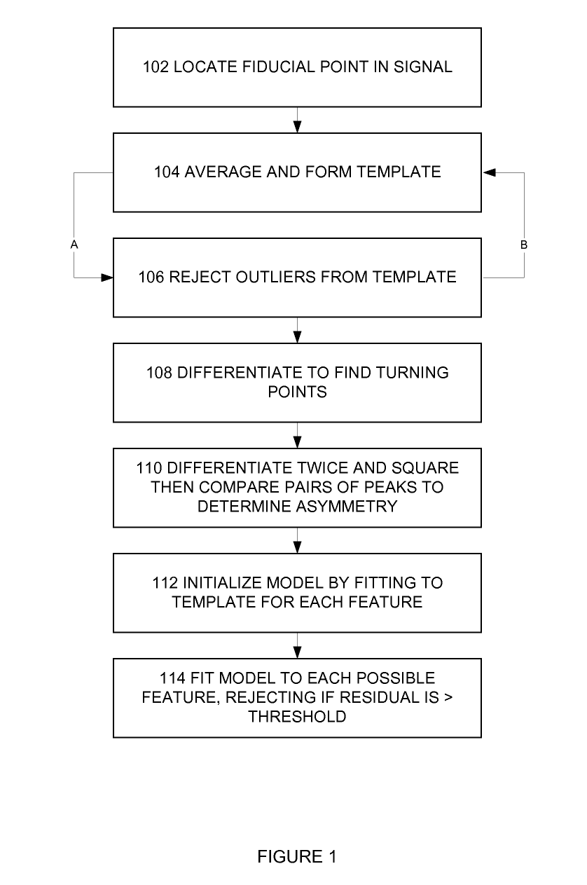

The flowchart of FIG. 1 shows a generalized method for constructing a model according to the invention. In particular, the method of the invention provides a general framework for deriving models of quasi-stationary signals for robust filtering, compression and segmentation of a signal and for identifying the location of regions of change. As such, the method can be viewed as a type of novel adaptive filter or as a method for correlated source separation in the time domain. In particular, the approach is suited to physiological signals, which are often characterized by oscillations at specific frequencies, and contaminated by in-band noise (which is both periodic and statistical).

The assumption in the following method is that the time series under analysis is composed of a set of distinct, yet transient (although not necessarily independent) morphologies. Examples of these include the set of features used to classify sleep from the electroencephalogram, (such as K complexes and sleep spindles), the heart sounds recorded in the phonocardiogram, or the waves in a pulsatile blood pressure waveform. Once a set of general features is identified, a template of each feature may be formed and a mixture of temporally shifted basis functions (such as Gaussians) may be fitted to each major turning point in the signal using an optimization procedure.

The signal model is a dynamic model, where each turning point in a signal is represented by a Gaussian of varying width and amplitude, centered at different points in time. This novel implementation extends the model by adding a new Gaussian for each asymmetric turning point, then adaptively modifying the parameters to fit a distinct observation. Here, the concept is generalized to model any signal and provide an automatic method for deriving the mode parameters.

If we assume a transient feature (such as a K complex) is smoothly varying and composed of M symmetric and N asymmetric turning points, then M+2N Gaussians are required to describe the feature (since a Gaussian is symmetric). For example, an asymmetric turning point requires two Gaussians to be accurately represented. The segment of the signal z, which describes the feature under analysis is given by:

$$z = {\sum }_{i = 1}^{M + 2 N} k exp ( Δ {t}_{i}^{2} / 2 {b}_{i}^{2} ) + {z}_{i} t \qquad ( 1 )$$

where Δtᵢ=(t−tᵢ), is the relative position of each turning point from the location in time t, of a reference point (fiducial marker), κ=aᵢ/2bᵢ is a normalization constant (chosen for consistency with the original dynamic model), and the zᵢ are baseline offset parameters for each of the turning points. The coefficients aᵢ govern the magnitude of the turning points and the bᵢ define the width (time duration) of each turning point. The model is therefore fully described by 3(M+2N) parameters.

In order to fit Equation (1) to a feature, an approximate template must be constructed. A general method for this is to apply a coarse matched filter (such as cross correlation with a population independent general template) or an energy thresholding technique (which is common in ECG analysis) to the signal in question. The selection of one technique for this process over another depends on the distribution of the energy of the observation over time. If the signal energy is evenly distributed over time, some a priori knowledge of the features may be used to form a simple template for a matched filter.

Fiducial markers may then be located at various points in time that provide time-specific reference markers for each candidate feature (segment of signal) as shown in step 102 of FIG. 1. By segmenting the time series around each fiducial point, and performing a temporal average, a first template class is generated as shown by step 104. By comparing each candidate feature to the first template class, possible artifacts or patterns belonging to other feature classes may be rejected using a suitable threshold such as a cross-correlation as shown in step 106.

The first feature class may then be modified to be the average of the non-rejected individual features (to construct a more specific feature). The rejected candidate may then be averaged to form a second feature class template and the process repeated (see arrows A and B) until the number of possible remaining candidates (which were not included in the previous classes) are below some pre-defined threshold, or the inter-pattern variance between the remaining candidate patterns becomes too high to allow the formation of any more distinct groups.

In the case of an ECG, the first feature class is likely to be a sinus beat (as long as it is the dominant morphology in the time series). Abnormal beats may be rejected and the dominant abnormal beat may become the second feature class. High correlations between the average of this rejected set and each member of the set may identify the new members of the set. Rejected beats may cascade down to the next candidate class.

For each template class, an initial model must then be derived. The model order O=M+2N, the number of symmetric plus twice the number of asymmetric turning points in the class. Often, this is a known quantity for most physiological features, but in some circumstances, an unsupervised method for determining the model order is required.

One method is as follows: if there are enough feature candidates to form a smooth, low noise template, the number of turning points may be calculated by numerically differentiating the feature and locating the zero crossing points (after allowing for delays in the numerical differentiation function) as shown in step 108.

The degree of asymmetry for each turning point may then be found by squaring the resultant differential and comparing the resultant two peaks (one for the upslope and one for the downslope) as shown in step 110. If a given pair of peaks are similar in height and width, then the peak is symmetric and only one set of aᵢ, bᵢ, and tᵢ are required for the peak. If the peaks in the squared differential, for a given pair, differ sufficiently (by some predefined threshold that depends on the feature class and signal amplitudes) then the peak is deemed asymmetric and two Gaussians are required to describe the turning point.

It should be noted that this procedure effectively determines the approximate starting points for fitting the model to each feature candidate (see step 112). However, the height (aᵢ) and width (bᵢ) of each Gaussian in the initial model remain to be determined. For most applications, (as long as the tᵢ are initially limited so that they do not vary significantly) the initialization of the aᵢ and bᵢ do not affect the final outcome, and random small values are sufficient. However, in some situations, abnormal local minima in the model fitting procedure are possible and the use of an estimate of the width and height of the turning points not only helps to avoid this, but also allows a significant acceleration in the time for fitting each feature candidate.

The residual error between the result of the model fitting procedure (described below) and the original feature provides a facility to reject particular fits as shown in step 114. It should also be noted that a classification may be performed by initializing with each possible class (variant of the model) and picking the class with the minimum residual error, or the smallest distance (in parameter space) between a given fit and a cluster center of representative candidates in the same parameter space.

An efficient method of fitting the signal model (Equation (1)) to a candidate vector s(t), is to minimize the squared error between s and the model output, z. In other words, one should find

$${ɛ}_{r} = {min}_{{a}_{i} , {b}_{i} , {θ}_{i}} {‖ s ( t ) - z ( t ) ‖}_{2}^{2}$$

over all of the 3(M+2N) parameters in the model. Equation (1) may be solved using an (3M+6N)-dimensional nonlinear gradient descent on the parameter space. In general, the problem of multidimensional nonlinear least squares fitting requires the minimization of the squared residuals of n functions, fⱼ, in p parameters, xⱼ,

$$\begin{matrix}Φ ( x ) = ( 1 / 2 ) {\sum }_{j = 1}^{n} {{f}_{j} ( {x}_{i} , … , {x}_{p} )}^{2} \\ = ( 1 / 2 ) {‖ F ( x ) ‖}^{2}\end{matrix}$$

all algorithms for achieving the minimization may proceed from an initial guess using the linearization,

ψ(p)=∥F(x+p)∥≈∥F(x)+Jp∥

where x is the initial point, p is the next step and J is the Jacobian matrix Jⱼₖ=dfⱼ|dxₖ. Additional strategies can be used to enlarge the region of convergence and include requiring a decrease in the norm ∥F∥ on each step or using a trust region to avoid steps that fall outside the linear regime. This procedure has been implemented in two different libraries: the Gnu Scientific Libraries (GSL) in C, and in Matlab using the function Isqnonlin.

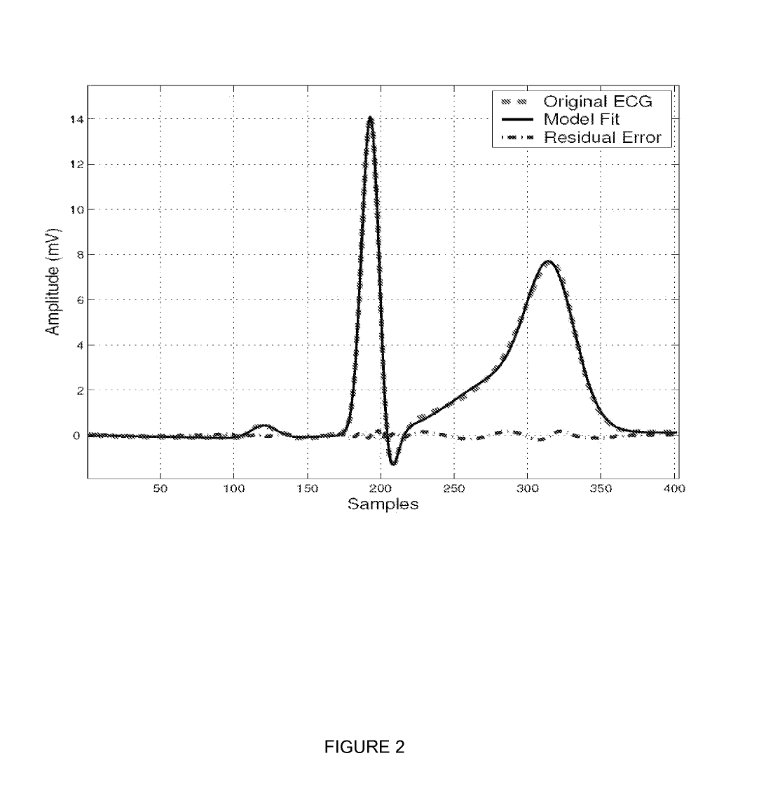

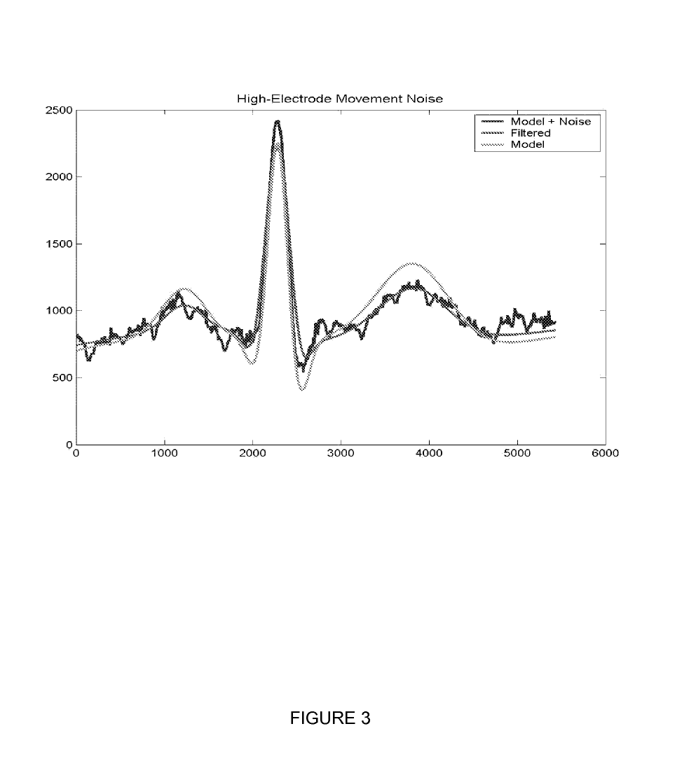

In one specific application, the invention may be applied in a novel technique for fitting a nonlinear ECG model (a sum of temporally shifted Gaussian waveform morphologies) to the ECG using a nonlinear least squares optimization. FIG. 2 illustrates the performance of the fitting procedure for a typical ECG with no noise in the original signal. FIG. 3 illustrates the performance of the technique when fitting the model to an extremely noisy beat. Not only does the technique allow a powerful method for filtering the ECG on a beat-by-beat basis even in high noise conditions, but also the use of Gaussian descriptors allows for a statistically meaningful description of wave onset and offset. In particular, this model-fitting procedure provides an excellent method for Q-wave onset and T-wave offset localization.

The model-based fitting of an ECG allows one to more precisely determine the locations of the P, Q, R, S and T features of each beat, and their respective onsets and offsets (determined as a certain number of standard deviations away from the central point). Furthermore, since noise may not be explicitly encoded in the waveform, the fitting procedure makes for an excellent noise suppression technique. Although the representation of the beat as just 18 coefficients in a nonlinear model means that (lossy) compression is possible, the clustering of these coefficients allows one to classify beats on this basis. However, perhaps the most useful and immediate application of this model-fitting procedure is in the determination of wave boundaries in noisy conditions to allow robust and accurate QT analysis.

Accordingly, by fitting a modified version of the model to each beat, and constraining the fit with a time averaged template, a filtering of each beat is performed. The model consists of a sum of Gaussians centered on each wave of the ECG (P, Q, R, S, and T). Each Gaussian is fully specified by three parameters: location in time, amplitude, and broadness. Therefore, the representation of the ECG as a series of Gaussians is also a form of (lossy) compression. Finally, the parameters for each beat may be compared to a normal set of parameters and a classification made.

Another way to apply the above-noted approach is to describe each feature of the ECG (PQRS & T) by a Gaussian with three parameters: the amplitude aᵢ, width bᵢ, and phase θᵢ=2π/tᵢ (or relative position with respect to the R-peak). The vertical displacement of the ECG, z, is described by an ordinary differential equation,

$$\dot{z} ( {a}_{i} , {b}_{i} , {θ}_{i} ) = - {\sum }_{i ∈ [ P , Q , R , S , {T}^{-} , {T}^{+} \}} {a}_{i} {Δθ}_{i} {e}^{\frac{- {Δθ}_{i}^{2}}{2 {b}_{i}^{2}}} \qquad ( 3 )$$

where Δθᵢ=(θ−θᵢ) is the relative phase. Note that no z-offset exists as the model-fit assumes z=0 at the isoelectric level. Numerical integration of this equation using appropriate set of aᵢ, bᵢ, θᵢ leads to the familiar ECG waveform.

Furthermore, an optional extra parameter has been added to the T feature, denoted by a superscripted − or +, to indicate that they are located at values of θ (or t) slightly either side of the original θᵢ. By using two sets of {a₁, bᵢ, θᵢ} to represent a particular feature, an asymmetric turning point may be formed. Although this is particularly important for the T-wave on the ECG, it is of negligible importance for the other four features in the ECG. Therefore, six features may be required for the ECG: P, Q, R, S, T⁻, T⁺.

Again, an efficient method of fitting the ECG model described above to an observation s(t), is to minimize the squared error between the s(t) and z. That is, one may find

$${min}_{{a}_{i} , {b}_{i} , {θ}_{i}} {‖ s ( t ) - z ( t ) ‖}_{2}^{2} \qquad ( 4 )$$

over all six i, with tᵢ=2π/θᵢ. Fortunately, one may analytically integrate (3) to give

$$\dot{z} ( {a}_{i} , {b}_{i} , {t}_{i} ) = \sum 2 {a}_{i} {Δθ}_{i} exp ( - {θ}_{i}^{2} / 2 {b}_{i}^{2} ) .$$

Equation (4) may then be solved using an eighteen-dimensional gradient descent in the parameter space. The Matlab function Isqnonlin.m or the like may perform the required implementation of this nonlinear least squares optimization.

To minimize the search space for fitting the parameters (a₁ᵢ, bᵢ, and θᵢ), a simple peak-detection and time-aligned averaging to form an average beat morphology template is formed over, for example, at least the first 60 beats centered on their R-peaks. (The template window is unimportant, as long as it contains all the PQRST features and does not extend into the next beat). Cross correlation is then performed between each beat and the template to remove outliers (with a linear cross-correlation coefficient less than, for example, 0.95). If more than about 20% of the beats are removed, then another 60 beats may be allowed into the average template, and the outlier rejection procedure is re-iterated. When less than about 20% of the beats are discarded, another average template is then made of the remaining beats. Peak and trough detection is then performed on this template (using refactory constraints for each wave) to find the relative locations of the turning points in time (and hence the θᵢ). The values T⁻ and T⁺ may be initialized ±40 ms either side of θ_{T}. By measuring the heights of each peak (or trough) an estimate of the aᵢ may also be made. Each bᵢ may be initialized with a value 10+5μ, where μ is a uniform distribution on the interval [0, . . . , 1]. Each of the values, aᵢ, and θᵢ, were initialized with random perturbations of μ and 20μ respectively.

Note that it is important that salient features that one wishes to fit (the P-wave and QRS segment in the case of the ECG) are sampled at a high enough frequency to allow them to contribute sufficiently to the optimization. In empirical tests, it has been found that all ECGs below approximately 512 Hz required upsampling (with an appropriate antialiasing filter). This corresponds to about 30 sample points in the QRS complex. Using less than 30 samples in a wave may lead to some extremely bizarre fits that fulfill the optimization criteria.

FIG. 2 shows an original (clean) graphed ECG signal, a model fit signal constructed according to the invention and the residual error between fit and model signals. In particular, FIG. 2 illustrates a real beat (recorded from a V5 lead), a typical fit to a template of real beat, and the residual error.

FIG. 3 illustrates the results of fitting the model to a segment of ECG cleanly recorded and contaminated by electrode motion noise. Note that despite the significant waveform distortion, the locations of the P, Q, R, S, and T peaks match the underlying (uncorrupted signal) to sub-sample precision, even with (Fₛ>1 kHz). Note also that the error around the iso-electric point and ST-level are negligible in a clinical sense (<0.1 mV, or about 5% to 10% of the QRS amplitude for a sinus beat on a V5 lead) (Amplitudes have been scaled by an arbitrary, but consistent factor).

Filtering of the ECG by fitting equation 3 to small segments of the ECG around each QRS-detection fiducial point is an excellent way to provide an idealistic (zero-noise) representation of the morphology that captures much of the clinical information of that beat. In fact, this approach may be generalized to any band-limited waveform with fewer than Fₛ oscillations per sample. In particular, the signal we are representing does not need to be periodic and is therefore particularly suited to physiological signals. Since the model is a compact representation of oscillatory signals with few turning points compared to the sampling frequency, it therefore has a band pass filtering effect leading to a lossy transformation of the data into a set of integrable Gaussians distributed over time.

It should be noted that that the fitting procedure effects a (lossy) compression at a rate of

$$( \frac{{F}_{s}}{3 k} : 1 )$$

per beat or

$$( \frac{\overline{RR}}{3} \frac{{F}_{s}}{3 k} : 1 ) ,$$

where R̅R̅ is the reciprocal of the (average) heart rate, Fₛ is the sampling frequency, and k=n+2m is the number of features or turning points used to fit the heart beat morphology (with n symmetric and m asymmetric turning points). For a low ECG sampling rate of 128 Hz, this translates into a compression ratio greater than 7:1 at a heart rate of 60 bpm. However, for high sampling rates (Fₛ=1024) this may lead to compression rates of almost 57:1. Reducing k from the full representation of k=18 is often appropriate for tasks which require only the QRS complex (k=9) or the ST segment (k=12) to be analyzed. High heart rates may reduce this compression unless the dynamic properties of the model are used to encode the heart rate-dependent variations through dynamic shifts in the values of the aᵢ, bᵢ, and θᵢ. For a given segment of τ seconds with an average heart rate of

$$\frac{60}{\overline{RR}} ,$$

the compression ratio rises by a factor

$$\frac{τ}{\overline{RR}} .$$

The above model is just an approximation and therefore the compression becomes even more lossy. One should also note that no explicit accounting of abnormal beats has been made in these calculations and a new set of parameters must be derived, possibly for each new abnormal beat encountered in the ECG record.

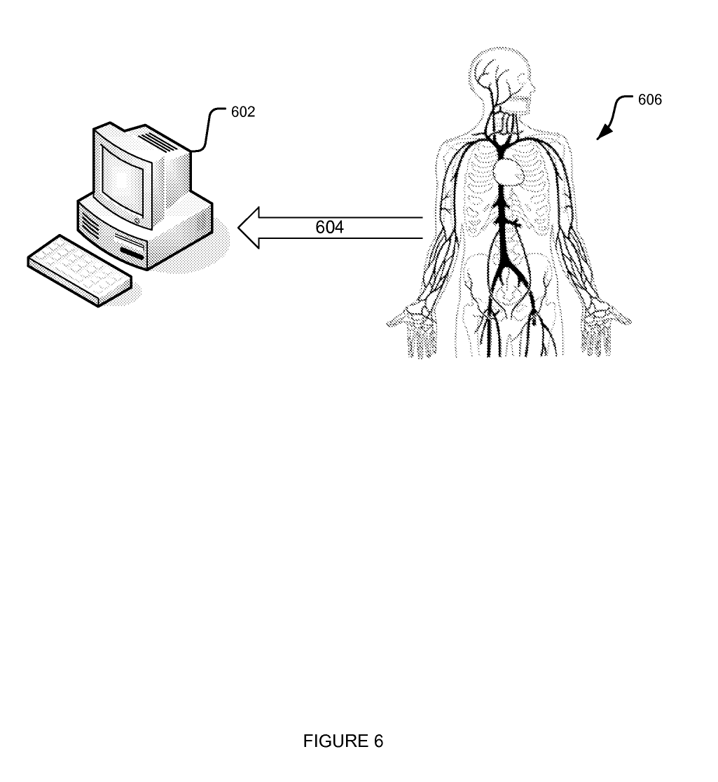

FIG. 6 shows a system constructed according to the principles of the invention for use with the method of the invention, such as FIG. 1. In particular, the above-described method for simultaneously filtering, compressing, and classifying a physiological signal 604, such as the ECG, from a subject 606 may work in real time on a modern desktop PC 602 and the like as shown in FIG. 6. The PC 602 may execute a signal processing program such as Matlab™ (Available from: The MathWorks, Inc., Natick, Mass. 01760-2098) or the like to perform the above-noted method as is known in the art. By fitting a set of six Gaussians, each specified by three parameters in an ordinary differential equation, and performing a constrained nonlinear optimization, it has been shown that in-band noise may be removed. One advantage of using prior knowledge concerning beat morphology is that a fitting error may be calculated with respect to the model, and thus we have an in-line measure of how well the procedure has filtered the ECG segment. By measuring the distance between the fitted parameters and pre-trained clusters in the 18-dimensional parameter space, classification is possible.

It should be noted that the real test of the filtering properties is not the residual error, but how distorted the clinical parameters of the ECG (such as the ST-level and QT interval) really are and whether they cause an abnormal beat to be erroneously classified as a normal beat. The methods of the invention produce insignificant distortion in clinical parameters for high levels of noise. For instance the model-based filter may introduce insignificant clinical distortion in the QT interval and QRS width down to an SNR≧0 dB for 1/f̂Beta noise for Beta<2. The fiducial point location may be insignificantly distorted (<1 ms) for an SNR≧2 dB, and the ST-level may be stable down to SNR>12 dB. The PR-interval may be more sensitive to noise due to the low amplitude nature of the P-wave, but still robust to noise. In general, the filter performance may be degraded by increasing Beta. See also the inventor's prior publications: “Method to Filter ECGs and Evaluate Clinical Parameter Distortion Using Realistic ECG Model Parameter Fitting,” G. D. Clifford, P. E. McSharry, Computers In Cardiology 2005, Sep. 25-28, 2005, pages 715-718; and “Model-based filtering, compression and classification of the ECG,” G. D. Clifford, A. Shoeb, P. E. McSharry, B. A. Janz: Invited paper for the Joint Meeting of 5th International Conference on Bioelectromagnetism and 5th International Symposium on Noninvasive Functional Source Imaging within the Human Brain and Heart (BEM&NFSI), Minnesota, May 12, 2005, which are incorporated by reference herein in their entirety.

The method of producing confidence intervals for a particular fit, or classification is an important step in determining the performance of a particular algorithm. In-line methods such as these may facilitate the robust interpretation of data and algorithms, reducing the number of false alarms that are triggered. In particular, the smooth nature of the fitted waveform allows for simple and robust detection of clinical features such as the iso-electric point, QT-interval, and ST level. The residual error from the fitting procedure then provides a confidence measure for the model-derived values of these features.

The above-described model has been generalized to allow modeling of turning points that exhibit asymmetries (such as the T-wave) by allowing such a feature to be described by two Gaussians. The model as such, may now be used to represent any waveform. However, the model complexity increases considerably for stochastic processes that inherently have many fluctuations compared to the sampling frequency. The main utility of the method detailed herein lies in the fact that the model represents smooth oscillations with few turning points compared to the sampling frequency, and therefore has a morphology-specific multi-band pass filtering effect leading to a lossy transformation of the data into a set of integrable Gaussians distributed over time. Each clinical feature of the ECG waveform is represented by a known and limited set of parameters. This allows for a very compact representation of the ECG morphology and makes the description mathematically tractable and completely generalizable to any semi-periodic signal.

Testing of the invention has resulted in accurate QT interval estimates. In contrast, it has been found that ECG analysts consistently pick the T offset to be early, since the analysts are unable to discern T-wave ends from the noise in the data. Accordingly, adaptation of the Gaussian model-based algorithm to locate Q-onset and T-offset points in a robust fashion, allows an accurate method for QT interval measurement, even in high noise situations.

It is further contemplated that the invention may utilize extra information with 12 leads with the use of a multi-channel QT analysis system, with noise rejection using Independent Component Analysis, Principal Component Analysis and Frank lead reconstruction (using the (inverse) Dower transform). By determining the noise content of each lead and using these dimensionality reduction techniques, the sensitivity of QT analysis to varying levels and types of noise may be evaluated, to provide a principled on-line confidence index for each QT interval evaluation. The relationship between the QT interval, preceding and following RR intervals, and other ECG model parameters (P, Q, R, S, and T amplitude and duration) such as U wave detection and characterization, T-wave height, and T-wave asymmetry are also contemplated by the invention.

The algorithm and analytic framework discussed above may also be adapted in the following ways:

if Bi-phasic QRS complex and P waves are employed, two Gaussians can be used either when there is a substantial asymmetry (skew greater than a given lead dependent threshold for the P wave or T wave) or when there are two significant peaks for each conventional point (P, Q, R, S, T, or U).

The asymmetry of each wave (T in particular) may be well modeled by a log-normal distribution. Therefore, other embodiments of this approach may consider log-normal distributions also. Disadvantages exist in that the probabilistic interpretation is not so well defined, but there are fewer parameters to fit.

QT interval determination may be made using probabilities—as well as using the zero-gradient criterion, to more accurately mimic the more conservative human tendency to under-estimate the end of repolarization using the M-sigma point of the two Gaussians in the T wave. This may be calculated using (for N Gaussians):

mu_{—}T=a_(N−1)*mu_(N−1)+a_{—}N*mu_{—}N

sigma_{—}T=(a_(N−1)̂2*sig_(N−1)̂2+a_{—}N̂2*sig_{—}N̂2)̂½.

and the end of the T wave is taken to be mu_T+M*sigma_T, where M=2 for most leads, but can take other values.

More sensitive QT analysis is also possible with the model of the invention. There may be considerable overlap in QT/QTc between normal and non-normal patient groups. See e.g., “The Spectrum of Symptoms and QT Intervals in Carriers of the Gene for the Long-QT Syndrome,” G. M. Vincent, K. W. Timothy, M. Leppert, M. Keating, N Engl J. Med. 1992; 327: 846-852. More sensitive measures of other repolarization-related properties may be used to reveal a more sensitive metric for classifying patients as normal or not normal, including:
Measure of T-wave amplitude or relative T-wave amplitude (such as the R-peak divided by T-wave peak height);
Asymmetry of the T-wave (such as the skewness of the jT segment);
Length of the jT segment (as measured by the 2 sigma point defined above: sigma_T=(a_(N−1)̂2*sig_(N−1)̂2+a_N̂2*sig_N̂2)̂½.
Peakiness of the T-wave (such as the kurtosis of the jT segment).

Short QT syndrome (SQTS) leads to an abbreviated QTc interval and predisposes patients to life-threatening arrhythmias. To date, three forms of the disease have been identified: SQT1, caused by a gain of function substitution in the HERG (IKr) channel, SQT2, caused by a gain of function substitution in the KvLQT1 (Iks) channel, and, SQT3, which has a unique ECG phenotype characterized by asymmetrical T waves. See, e.g. “A Novel Form of Short QT Syndrome (SQT3) Is Caused by a Mutation in the KCNJ2 Gene,” Silvia G. Priori, Sandeep V. Pandit, Ilaria Rivolta, Omer Berenfeld, Elena Ronchetti, Amit Dhamoon, Carlo Napolitano, Justus Anumonwo, Marina Raffaele di Barletta, Smitha Gudapakkam, Giuliano Bosi, Marco Stramba-Badiale, and Jose Jalife, Circ. Res. 96: 800-807. Therefore, the above discussion of height, skew, width and kurtosis variables as above may be used in a SQTS application to help improve the sensitivity of short QT analysis significantly.

QTd (QT dispersion) is defined as the difference between the maximum and minimum QT intervals of any of 12 leads. QTd is sometimes thought to be a marker of myocardial electrical instability and has been proposed as a marker of the risk of death for those awaiting heart transplantation. See e.g., “Development of Automated 12-Lead QT Dispersion Algorithm for Sudden Cardiac Death,” M. B. Malarvili, S. Hussain, Ab. Rahim Ab. Rahman, The Internet Journal of Medical Technology, 2005, Volume 2 Number 2. In a similar way to QT intervals, QTd takes a Gaussian histogram of values for a particular population. There is a significant cross-over between normal and those at risk of sudden cardiac death (SCD). The mean value of QTd±SD is 37.28±11.13 ms (p<0.05) for a non-MI group and 66.17±13.95 ms (p<0.05) for the MI group. With QTd<50 ms is the threshold for normality, but this would lead to 20-30% of the normals being classified as MI and ˜20% being classified as non-MI. Using the height, skew, width and kurtosis variables as above would improve the sensitivity significantly.

The algorithm and analytic framework discussed above may also be used to perform very sensitive analysis of any feature of an ECG, including to:

filter ECGs

compress ECGs for efficient storage, transmission, and reconstruction

perform P-wave detection (and hence atrial beat/rhythm classification)

perform amplitude and QRS axis analysis (for deriving respiration)

perform beat classification (from clustering the parameters)

perform robust QT interval analysis

perform robust ST-segment analysis

perform PQRST subtraction for high frequency QRS analysis for diagnosis of ischemic heart disease, respiration related issues, and the like

perform PQRST subtraction for late potential analysis

perform T-wave alternan classification

perform rhythm analysis

In accordance with various embodiments of the invention, the methods described herein are intended for operation with dedicated hardware implementations including, but not limited to, PCs, PDAs, semiconductors, application specific integrated circuits (ASIC), programmable logic arrays, and other hardware devices constructed to implement the methods described herein. Moreover, various embodiments of the invention described herein are intended for operation as software programs running on a computer processor. Furthermore, alternative software implementations including, but not limited to, distributed processing, component/object distributed processing, parallel processing, virtual machine processing, any future enhancements, or any future protocols thereof may also be used to implement the methods described herein.

It should also be noted that the software implementations of the invention as described herein are optionally stored on a tangible storage medium, such as: a magnetic medium such as a disk or tape; a magneto-optical or optical medium such as a disk; or a solid state medium such as a memory card or other package that houses one or more read-only (non-volatile) memories, random access memories, or other re-writable (volatile) memories. A digital file attachment to email or other self-contained information archive or set of archives is considered a distribution medium equivalent to a tangible storage medium. Accordingly, the invention is considered to include a tangible storage medium or distribution medium, as listed herein and including art-recognized equivalents and successor media, in which the software implementations herein are stored.

While the invention has been described in terms of exemplary embodiments, those skilled in the art will recognize that the invention can be practiced with modifications in the spirit and scope of the appended claims. These examples given above are merely illustrative and are not meant to be an exhaustive list of all possible designs, embodiments, applications, or modifications of the invention. For example, the invention may fit a set of alternate basis functions to the signal, perhaps using some other form of optimization; may use other signals other than physiological signals; may use any set of basis functions, not just Gaussians; may use any optimization routine to fit the basis functions to the observation—least squares, nonlinear least squares, gradient descent with any cost function and any activation function (such as tanh or softmax in a neural network). Moreover, IIR/FIR filters, independent Component Analysis (ICA); Principal Component Analysis (PCA) I Singular Value Decomposition (SVD)/Karhunen Loeve Transform (KLT)/Hotelling Transform; Auto-Regressive (AR) modeling—equivalent to Fourier Transform; and Wavelet Analysis (Laguna et al, Hughes et al.) approaches may also be used for further pre-processing or post-processing.

What is claimed:

1. A computer-implemented method for at least one of filtering, segmenting, compressing, and classifying an ECG signal comprising the steps of:
obtaining an ECG signal;
storing the ECG signal;
generating a nonlinear signal model based on the ECG signal;
fitting the nonlinear signal model to the ECG signal based on an optimization algorithm;
determining at least one feature of the ECG with the nonlinear signal model; and
outputting the at least one feature of the ECG based on the nonlinear signal model.

2. The method according to claim 1 wherein the step of generating a nonlinear signal model corresponds to modeling at least one segment of interest of the ECG signal selected from the group consisting of a QT interval, a Q-wave onset (PQ-junction), a T-wave offset, a T-wave height, a U-wave detection and characterization, and a T-wave asymmetry of the ECG signal.

3. The method according to claim 1 wherein said generating step comprises the use of Gaussian descriptors.

4. The method according to claim 1 wherein the optimization algorithm in said fitting step comprises least squares optimization.

5. The method according to claim 1 wherein said generating step comprises the use of Gaussian descriptors and the optimization algorithm comprises least squares optimization.

6. The method according to claim 1 wherein said step of determining at least one feature comprises determining at least one of a QT interval, a Q-wave onset (PQ-junction), a T-wave offset, a T-wave height, a U-wave detection and characterization, and a T-wave asymmetry of the ECG signal.

7. The method of claim 6 wherein said step of determining at least one feature comprises determining each one of a QT interval, a Q-wave onset (PQ-junction), a T-wave offset, a T-wave height, a U-wave detection and characterization, and a T-wave asymmetry of the ECG signal.

8. The method according to claim 1 wherein said step of determining at least one feature comprises determining the locations of the P, Q, R, S and T features of each beat of the ECG signal.

9. The method according to claim 1 wherein said generating step further comprises the steps of:
locating at least one fiducial point in the ECG signal;
performing a temporal average of time series segments around the at least one fiducial point;
accepting features inside a threshold; and
determining the symmetry of the features that are accepted.

10. The method according to claim 9 wherein said generating step further comprises the steps of:
fitting the model to the features; and
rejecting model fit when the model exceeds a threshold.

11. An adaptive filter using the method of claim 1.

12. The filter of claim 11, wherein said filter operates on a beat-by-beat basis.

13. A computer readable medium executable on a computer for at least one of filtering, segmenting, compressing, and classifying an oscillatory physiological signal, the computer readable medium executing the steps of:
obtaining an ECG signal;
storing the ECG signal;
generating a nonlinear signal model based on the ECG signal;
fitting the nonlinear signal model to the ECG signal based on an optimization algorithm;
determining at least one feature of the ECG with the nonlinear signal model; and
outputting the at least one feature of the ECG based on the nonlinear signal model.

14. The computer readable medium according to claim 13 wherein the step of generating a nonlinear signal model corresponds to modeling at least one segment of interest of the ECG signal selected from the group consisting of a QT interval, a Q-wave onset (PQ-junction), a T-wave offset, a T-wave height, a U-wave detection and characterization, and a T-wave asymmetry of the ECG signal.

15. The computer readable medium according to claim 13 wherein said step of generating comprises the use of Gaussian descriptors.

16. The computer readable medium according to claim 13 wherein the optimization algorithm in said fitting step comprises least squares optimization.

17. The computer readable medium according to claim 13 wherein said generating step comprises the use of Gaussian descriptors and the optimization algorithm comprises least squares optimization.

18. The computer readable medium according to claim 13 wherein said step of determining at least one feature comprises determining at least one of a QT interval, a Q-wave onset (PQ-junction), a T-wave offset, a T-wave height, a U-wave detection and characterization, and a T-wave asymmetry of the ECG signal.

19. The method of claim 18 wherein said step of determining at least one feature comprises determining each one of a QT interval, a Q-wave onset (PQ-junction), a T-wave offset, a T-wave height, a U-wave detection and characterization, and a T-wave asymmetry of the ECG signal.

20. The computer readable medium according to claim 13 wherein said step of determining at least one feature comprises determining the locations of the P, Q, R, S and T features of each beat of the ECG signal.

21. The computer readable medium according to claim 13 wherein said generating step further comprises the steps of:
locating at least one fiducial point in the ECG signal;
performing a temporal average of time series segments around the at least one fiducial point;
accepting features inside a threshold; and
determining the symmetry of the features that are accepted.

22. The computer readable medium according to claim 21 wherein said generating step further comprises the steps of:
fitting the model to the features; and
rejecting model fit when the model exceeds a threshold.

23. A computer system for at least one of filtering, segmenting, compressing and classifying an oscillatory physiological signal, the computer system comprising:
an input to receive an ECG signal;
a storage device responsive to the input to store the ECG signal;
a processor to generate a nonlinear signal model based on the ECG signal, fit the nonlinear signal model to the ECG signal based on an optimization algorithm, and determine at least one feature of the ECG with the nonlinear signal model; and
an output device to output the at least one feature of the ECG based on the nonlinear signal model.

24. The computer system according to claim 23 wherein the nonlinear signal model corresponds to at least one segment of interest of the ECG signal.

25. The computer system according to claim 23 wherein the nonlinear signal model comprises Gaussian descriptors.

26. The computer system according to claim 23 wherein the optimization algorithm comprises least squares optimization.

27. The computer system according to claim 23 wherein the nonlinear signal model comprises Gaussian descriptors and the optimization algorithm comprises least squares optimization.

28. The computer system according to claim 23 wherein the at least one feature comprises at least one of a QT interval, a Q-wave onset (PQ-junction), a T-wave offset, a T-wave height, a U-wave detection and characterization, and a T-wave asymmetry of the ECG signal.

29. The computer system according to claim 23 wherein the at least one feature comprises at least one of the locations of the P, Q, R, S and T features of each beat of the ECG signal.

30. A computer system for at least one of filtering, segmenting, compressing and classifying an oscillatory physiological signal, the computer system comprising:
means for receiving an ECG signal;
means for storing the ECG signal;
means for generating a nonlinear signal model based on the ECG signal, fitting the nonlinear signal model to the ECG signal based on an optimization algorithm, and determining at least one feature of the ECG with the nonlinear signal model; and
means for outputting the at least one feature of the ECG based on the nonlinear signal model.

31. The computer system according to claim 30 wherein the nonlinear signal model corresponds to at least one segment of interest of the ECG signal.

32. The computer system according to claim 30 wherein the nonlinear signal model comprises Gaussian descriptors.

33. The computer system according to claim 30 wherein the optimization algorithm comprises least squares optimization.

34. The computer system according to claim 30 wherein the nonlinear signal model comprises Gaussian descriptors and the optimization algorithm comprises least squares optimization.

35. The computer system according to claim 30 wherein the at least one feature comprises at least one of a QT interval, a Q-wave onset (PQ-junction), a T-wave offset, a T-wave height, a U-wave detection and characterization, and a T-wave asymmetry of the ECG signal.

36. The computer system according to claim 30 wherein the at least one feature comprises at least one of the locations of the P, Q, R, S and T features of each beat of the ECG signal.
